(19) United States
(12) Patent Application Publication
(10) Pub. No.: US 2025/0368586 A1
(43) Pub. Date: Dec. 4, 2025

(54) PROCESSES FOR CONVERTING UNSATURATED POLYETHYLENE TO ALKENE PRODUCTS

(71) Applicants: Dow Global Technologies LLC, Midland, MI (US); The Regents of the University of California, Oakland, CA (US); The Board of Trustees of the University of Illinois, Urbana, IL (US)

(72) Inventors: Ivan A. Konstantinov, Manvel, TX (US); Rafael Huacuja, Rosharon, TX (US); Alex J. Nett, Midland, MI (US); Mari S. Rosen, Houston, TX (US); Sean W. Ewart, Pearland, TX (US); Garrett Strong, Goleta, CA (US); Lijun Gao, Santa Barbra, CA (US); Nicholas Wang, Pittsburg, PA (US); Vanessa DaSilva, Urbana, IL (US); Damien S. Guironnet, Urbana, IL (US); Susanna Scott, Goleta, CA (US)

(73) Assignees: Dow Global Technologies LLC, Midland, MI (US); The Regents of the University of California, Oakland, CA (US); The Board of Trustees of the University of Illinois, Urbana, IL (US)

(21) Appl. No.: 18/875,506

(22) PCT Filed: Jun. 14, 2023
(86) PCT No.: PCT/US2023/068417

§ 371 Date: Dec. 16, 2024

Related U.S. Application Data
(60) Provisional application No. 63/353,327, filed on Jun. 17, 2022.

(51) Int. Cl. C07C 4/06

(52) U.S. Cl. CPC C07C 4/06

(57) ABSTRACT

This disclosure relates to processes for converting unsaturated polyethylene to at least an alkene product. The processes comprise contacting the unsaturated polyethylene with two or more catalyst components in a reactor comprising an alkene reactant. The two or more catalyst components comprise a metathesis catalyst component and an isomerization catalyst component. Contacting causes at least a portion of the unsaturated polyethylene, or products derived therefrom, to undergo metathesis reactions and isomerization reactions to produce an effluent comprising at least the alkene product.

### CROSS-REFERENCE TO RELATED APPLICATIONS

This application claims the benefit of U.S. Provisional Application Ser. No. 63/353,327 filed Jun. 17, 2022, the entire disclosure of which is hereby incorporated herein by reference.

### BACKGROUND

### Field

The present disclosure relates to chemical processing of hydrocarbons. In particular, the present disclosure relates to processes for converting ethylene-containing materials, such as polyethylene into smaller desirable hydrocarbon products.

### Technical Background

For a number of industrial applications, hydrocarbons are used, or are starting materials used, to produce plastics, fuels, and various downstream chemicals. Such hydrocarbons include alkenes, such as ethene, propene and butenes (also commonly referred to as ethylene, propylene, and butylenes, respectively). A variety of processes for producing these lower hydrocarbons have been developed, including petroleum cracking and various synthetic processes. Polyethylene (PE), the most widely used plastic in the world, can be made into a wide variety of products. However, processes for the recycling of polyethylene into smaller monomers, such as propylene, is desired. Conventional efforts for chemical recycling of polyethylene have generally used pyrolysis and high-temperature thermal degradation. These processes are highly energy intensive and are plagued by low selectivity of desired products and generation of greenhouse gases (e.g. CO₂, CH₄).

### SUMMARY

Embodiments of the present disclosure address these and other needs by providing processes for converting polyethylene into alkene products. The processes described herein may enable two or more catalyst components in a reactor to conduct a plurality of different chemical reactions, such as combinations of metathesis and isomerization for producing alkene products from unsaturated polyethylene and an alkene reactant, for example.

According to one or more other aspects of the present disclosure, a process for converting unsaturated polyethylene to at least an alkene product of chemical formula CₘH₂ₘ, the process comprising contacting the unsaturated polyethylene with two or more catalyst components in a reactor, the reactor comprising an alkene reactant of chemical formula CₙH₂ₙ, where m is an integer from 3 to 20 and n is an integer from 2 to 20. The two or more catalyst components comprise a metathesis catalyst component and an isomerization catalyst component. Contacting causes at least a portion of the unsaturated polyethylene, or products derived therefrom, to undergo metathesis reactions and isomerization reactions to produce an effluent comprising at least the alkene product of chemical formula CₘH₂ₘ.

Additional features and advantages will be set forth in the detailed description which follows, and in part will be readily apparent to those skilled in the art from that description or recognized by practicing the embodiments described herein, including the detailed description which follows and the claims.

It is to be understood that both the foregoing general description and the following detailed description describe various embodiments and are intended to provide an overview or framework for understanding the nature and character of the claimed subject matter.

### BRIEF DESCRIPTION OF THE DRAWING

FIG. 1 schematically depicts a reactor, in accordance with one or more embodiments of the present disclosure.

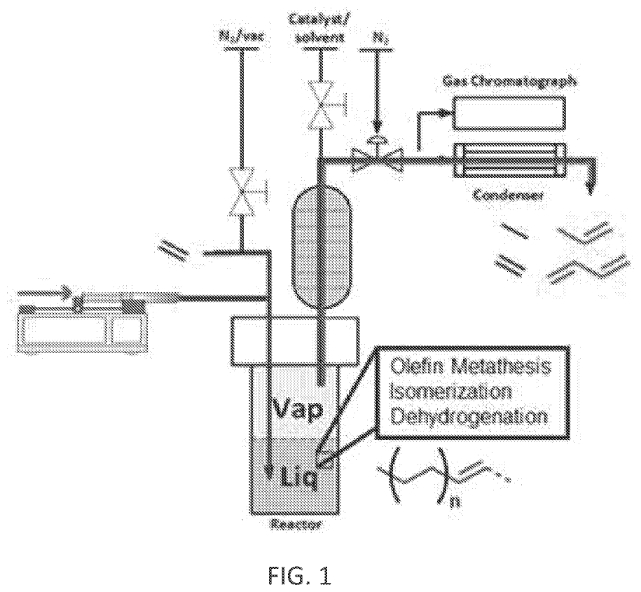

For the purpose of describing the simplified schematic illustration and description of FIG. 1, the numerous valves, temperature sensors, electronic controllers, and the like that may be employed and well known to those of ordinary skill in the art of certain chemical processing operations are not included. Further, accompanying components that are often included in typical chemical processing operations, carrier gas supply systems, pumps, compressors, furnaces, or other subsystems are not depicted. It should be understood that these components are within the spirit and scope of the present embodiments disclosed. However, operational components, such as those described in the present disclosure, may be added to the embodiments described in this disclosure

### DETAILED DESCRIPTION

Some conventional processes for converting polyethylene to smaller products may use separate catalysts isolated in separate catalyst zones, such as by charging each of the separate catalysts to a separate reactor, which can increase the initial capital cost of the reaction system. In contrast, processes disclosed herein can enable tandem catalysis of polyethylene by contacting the polyethylene with mutually compatible catalyst components to produce the desired alkene products. The catalytic depolymerization of polyethylene under mild reaction conditions provides an advantageous and sustainable alternative for the production of hydrocarbon feedstock, monomers or other useful chemicals.

Reference will now be made in detail to embodiments of processes for converting unsaturated polyethylene to alkene products in a reactor. As used herein, “unsaturated polyethylene” refers to a compound comprising the chemical formula CₓH₂ₓ, where x is an integer of at least 10, and where there is a least one carbon-carbon double bond. In embodiments, the unsaturated polyethylene can include branched polyethylene. In embodiments, the unsaturated polyethylene can include linear low-density polyethylene (LLDPE), low density polyethylene (LDPE), or combinations thereof. In embodiments, the unsaturated polyethylene comprises CₓH₂ₓ, where x is an integer of greater than or equal to 10, greater than or equal to 12, or even greater than or equal to 15. In embodiments, the unsaturated polyethylene can have a number average molecule weight (Mₙ) of from 150 g/mol to 1,000,000 g/mol. In embodiments, the unsaturated polyethylene can be a waste stream, or product derived therefrom, of a hydrocarbon processing system.

In embodiments, the reactor comprises an alkene reactant. In embodiments, the alkene reactant has a chemical formula of CₙH₂ₙ, where n is an integer from 2 to 20. For example, the alkene reactant can have a chemical formula of CₙH₂ₙ, where n is an integer from 2 to 15, from 2 to 10, from 2 to 5, from 2 to 4, or from 2 to 3. In embodiments, the alkene reactant can comprise ethylene, propylene, butenes, pentenes, or combinations thereof. In embodiments, the alkene reactant can be selected from the group consisting of ethylene, propylene, butenes, pentenes, and combinations thereof. In embodiments, the alkene reactant can comprise ethylene. In embodiments, the alkene reactant can consist essentially of or consist of ethylene. In embodiments, the alkene reactant can comprise ethylene and butenes. In embodiments, the alkene reactant can consist essentially of or consist of ethylene and butenes.

In embodiments, the unsaturated polyethylene can be contacted with two or more catalyst components in a reactor. In other embodiments, the unsaturated polyethylene can be contacted with three or more catalyst components in a reactor. As used herein, “catalyst components” refers to any substance which increases the rate of a specific chemical reaction. Catalyst components and the catalyst compositions made with the catalyst components described in this disclosure may be utilized to promote various reactions, such as, but not limited to, dehydrogenation, metathesis, isomerization, or combinations of these. In embodiments, a catalyst composition can include at least one catalyst component or at least two catalyst components. As used herein, “catalyst composition” refers to a solid particulate comprising at least one catalyst component. The catalyst composition can further comprise a catalyst support material.

In embodiments the catalyst components can include a metathesis catalyst component, an isomerization catalyst component, and optionally, a dehydrogenation catalyst component. Without intending to be bound by any particular theory, it is believed that the metathesis catalyst, in the presence of the alkene reactant, can break the carbon chain of the unsaturated polyethylene to produce two products that each have a terminal unsaturation, and further metathesis of the terminally unsaturated polyethylene intermediate product with the alkene reactant may be unproductive to further break the carbon chain. It is believed that the isomerization catalyst component can convert the terminal unsaturation to an internal unsaturation, and the isomerized product can be further broken into two products in the presence of the metathesis catalyst component and the alkene reactant. This cycle can continue until the desired product or group of products is produced from the process. Further, it is believed that the dehydrogenation catalyst can introduce additional unsaturations in the carbon chain of the unsaturated polyethylene, or products derived therefrom, which may increase the depolymerization of the unsaturated polyethylene.

In embodiments, the metathesis catalyst component in combination with the alkene reactant, such as ethylene, can be operable to break the unsaturated polyethylene chain into two species. In embodiments, the metathesis catalyst component can break alkene products derived from the unsaturated polyethylene. In embodiments, the metathesis catalyst component can include one or more elements selected from International Union of Pure and Applied Chemistry (IUPAC) groups 5-10. In embodiments, the metathesis catalyst component can comprise rhenium, ruthenium, tungsten, molybdenum, vanadium, or combinations thereof. In embodiments, the metathesis catalyst component can be selected from the group consisting of rhenium, ruthenium, tungsten, molybdenum, vanadium, and combinations thereof. In embodiments, the metathesis catalyst component can comprise methyltrioxorhenium (MTO).

In embodiments, the isomerization catalyst component can be operable to move an unsaturation on unsaturated polyethylene, or an unsaturation on products derived therefrom, from one position on the backbone to a different position. For instance, in embodiments, the isomerization catalyst component can move an unsaturation in a terminal position of the unsaturated polyethylene to an internal position. In embodiments, the isomerization catalyst component can include one or more elements selected from International Union of Pure and Applied Chemistry (IUPAC) groups 5-10. In embodiments, the isomerization catalyst component can comprise alumina, silica, iridium, palladium, ruthenium or combinations thereof. In embodiments, the isomerization catalyst component can be selected from the group consisting of alumina, silica, iridium, palladium, ruthenium, and combinations thereof. In embodiments, the isomerization catalyst component can include modified alumina, modified silica, or combinations thereof. For instance, in embodiments, the isomerization catalyst component can include, but not be limited to, chlorinated alumina, gamma-alumina, chlorinated silica, or combinations thereof. In embodiments, the isomerization catalyst component can comprise [tert-butyl-POCOP]Ir[C₂H_{4].}

In embodiments, the dehydrogenation catalyst component can cause unsaturated polyethylene, or products derived therefrom, to have additional unsaturations along the polyethylene backbone. In embodiments, the dehydrogenation catalyst component can cause the unsaturated polyethylene or products derived therefrom to undergo transfer dehydrogenation. In embodiments, the dehydrogenation catalyst component can include one or more elements selected from International Union of Pure and Applied Chemistry (IUPAC) groups 5-10. In embodiments, the dehydrogenation catalyst component can comprise platinum, iridium, ruthenium, rhenium, or combinations thereof. In embodiments, the dehydrogenation catalyst component is selected from the group consisting of platinum, iridium, ruthenium, rhenium, and combinations thereof.

In embodiments, the reactor may comprise one or more catalyst compositions that comprise the two or more catalyst components. For instance, in embodiments, a catalyst composition can comprise a metathesis catalyst component and an isomerization catalyst component. In embodiments, a catalyst composition can comprise a dehydrogenation catalyst component and an isomerization catalyst component. In embodiments, a catalyst composition can comprise a metathesis catalyst component and an isomerization catalyst component. In embodiments, a catalyst composition can comprise a dehydrogenation catalyst component and a metathesis catalyst component. In embodiments, a catalyst composition can comprise a dehydrogenation catalyst component, a metathesis catalyst component, and an isomerization catalyst component. In embodiments, a catalyst composition can comprise a dehydrogenation catalyst component, a metathesis catalyst component, or an isomerization catalyst component. In embodiments, the reactor can comprise a first catalyst composition comprising a metathesis catalyst component and an isomerization catalyst component. For instance, in embodiments, the reactor can comprise a first catalyst component, where the first catalyst component is MTO on alumina. In embodiments, the reactor can comprise a second catalyst composition comprising an additional metathesis catalyst component and/or isomerization catalyst component. In embodiments, the reactor can comprise a second catalyst composition comprising a dehydrogenation catalyst component and an isomerization catalyst component. For instance, in embodiments the reactor can comprise a second catalyst component, where the second catalyst component can comprise platinum on alumina or platinum on silica. In embodiments, a first catalyst composition comprising MTO on alumina and a second catalyst composition can contact the unsaturated polyethylene in the reactor.

In embodiments, the catalyst composition is designated by a weight percentage of the one or more elements selected from International Union of Pure and Applied Chemistry (IUPAC) groups 5-10. In embodiments, the first catalyst composition can comprise less than or equal to 15 wt. % of any one of the elements selected from the IUPAC groups 5-10 based on the total weight of the first catalyst composition. For instance, in embodiments, the first catalyst composition can comprise less than or equal to 12 wt. %, less than or equal to 10 wt. %, less than or equal to 8 wt. %, less than or equal to 6 wt. %, less than or equal to 4 wt. %, or even less than or equal to 2 wt. % of any one of the elements selected from the IUPAC groups 5-10 based on the total weight of the first catalyst composition. In embodiments, the first catalyst composition can comprise greater than 1 wt. %, greater than 2 wt. %, greater than 3 wt. %, greater than 4 wt. %, greater than 5 wt. %, greater than 6 wt. %, greater than 7 wt. %, greater than 8 wt. %, or even greater than 9 wt. % of any one of the elements selected from the IUPAC groups 5-10 based on the total weight of the first catalyst composition. In embodiments, the first catalyst composition can comprise any one of the elements selected from the IUPAC groups 5-10 in an amount of from 1 wt. % to 15 wt. %, from 1 wt. % to 12 wt. %, from 1 wt. % to 10 wt. %, from 1 wt. % to 5 wt. %, from 1 wt. % to 4 wt. %, from 2 wt. % to 15 wt. %, from 2 wt. % to 12 wt. %, from 2 wt. % to 10 wt. %, from 2 wt. % to 5 wt. %, from 2 wt. % to 4 wt. %, from 5 wt. % to 15 wt. %, from 5 wt. % to 12 wt. %, or from 5 wt. % to 10 wt. % based on the total weight of the first catalyst composition.

In embodiments, the second catalyst composition can comprise less than or equal to 15 wt. % of any one of the elements selected from the IUPAC groups 5-10 based on the total weight of the second catalyst composition. For instance, in embodiments, the second catalyst composition can comprise less than or equal to 12 wt. %, less than or equal to 10 wt. %, less than or equal to 8 wt. %, less than or equal to 6 wt. %, less than or equal to 4 wt. %, or even less than or equal to 2 wt. % of any one of the elements selected from the IUPAC groups 5-10 based on the total weight of the second catalyst composition. In embodiments, the second catalyst composition can comprise greater than 1 wt. %, greater than 2 wt. %, greater than 3 wt. %, greater than 4 wt. %, greater than 5 wt. %, greater than 6 wt. %, greater than 7 wt. %, greater than 8 wt. %, or even greater than 9 wt. % of any one of the elements selected from the IUPAC groups 5-10 based on the total weight of the second catalyst composition. In embodiments, the second catalyst composition can comprise any one of the elements selected from the IUPAC groups 5-10 in an amount from 1 wt. % to 15 wt. %, from 1 wt. % to 12 wt. %, from 1 wt. % to 10 wt. %, from 1 wt. % to 5 wt. %, from 1 wt. % to 4 wt. %, from 2 wt. % to 15 wt. %, from 2 wt. % to 12 wt. %, from 2 wt. % to 10 wt. %, from 2 wt. % to 5 wt. %, from 2 wt. % to 4 wt. %, from 5 wt. % to 15 wt. %, from 5 wt. % to 12 wt. %, or from 5 wt. % to 10 wt. % based on the total weight of the second catalyst composition.

It should be understood that according to embodiments, the catalyst composition may be made by methods that lead to the desired composition. Some non-limiting instances include incipient wetness impregnation, or vapor phase deposition of metal precursors (either organic or inorganic in nature), followed by their controlled decomposition.

In embodiments, contacting the unsaturated polyethylene with two or more catalyst components in a reactor comprising an alkene reactant can cause at least a portion of the unsaturated polyethylene, or products derived therefrom, to undergo metathesis reactions and isomerization reactions to produce an effluent comprising at least the alkene product of chemical formula CₘH₂ₘ. For instance, in embodiments, the unsaturated polyethylene can contact the metathesis catalyst component in the presence of the alkene reactant to break the unsaturated polyethylene to form two products, where each product comprises a terminal unsaturated polyethylene. The terminal unsaturated polyethylene can contact the isomerization catalyst component to cause the unsaturation to move in the terminal unsaturated polyethylene from a terminal position to an internal position to form an internal unsaturated polyethylene. Without intending to be bound by any particular theory, it is believed that the internal unsaturated polyethylene can undergo further metathesis reactions by contacting the metathesis catalyst component in the presence of the alkene reactant. It is believed that the products derived from the unsaturated polyethylene that contact both the metathesis catalyst component and the isomerization catalyst component in the presence of the alkene reactant can continue to cycle between metathesis and isomerization reactions to produce smaller alkene products, such as compounds of chemical formula CₘH₂ₘ, where m is an integer from 3 to 20, for instance, propylene. In embodiments, the reaction time can be increased to produce an effluent comprising smaller alkene products, as increased reaction time will allow additional metathesis and isomerization reaction cycles.

In embodiments, the reactor can be any reactor useful for causing the polyethylene to contact the two or more catalyst components in the presence of the alkene reactant and cause the catalytic reactions to proceed, such as a batch reactor, a fixed-bed reactor, a fluidized bed reactor, a continuous stirred tank reactor, a tubular plug flow reactor, a reactive extruder, or combinations thereof. In embodiments two or more reactors can be used, such as two or more reactors in series. In embodiments, the reactor can comprise a reaction zone where the contacting and the catalytic reactions can occur. In embodiments, the two or more catalyst components can be in the same reaction zone. In other embodiments, the reactor can comprise two or more reaction zones. In embodiments, the reactor can include additional processing of the reactants, such as processing of the alkene reactant, the unsaturated polyethylene, and/or the catalyst components. In embodiments, the effluent comprising one or more products from the catalytic reactions can be further processed, such as separation of one or more products from the effluent. For instance, in embodiments, propylene can be separated from the effluent.

In embodiments, a pressure of the alkene reactant in the reactor, such as in the reaction zone during the contacting can be from 0 pounds per square inch gauge (psig) to 3000 psig. For instance, a pressure of the alkene reactant can be of from 0 psig to 3000 psig, from 0 psig to 2000 psig, from 0 psig to 1000 psig, from 0 psig to 900 psig, from 0 psig to 800 psig, from 0 psig to 700 psig, from 0 psig to 600 psig, from 0 psig to 500 psig, or from 100 psig to 3000 psig. In some embodiments, the amount of the alkene reactant used can be quantified by the pressure of the alkene reactant in the reactor. In other embodiments, the amount of the alkene reactant can be quantified by a space velocity of the alkene reactant.

In embodiments, a temperature of the reactor, such as in the reaction zone, during the contacting can be less than or equal to 400° C. For instance, a temperature of the reactor during the contacting can be less than or equal to 350° C., less than or equal to 300° C., less than or equal to 250° C., or even less than or equal to 200° C. In embodiments, a temperature of the reactor during the contacting can be of from 50° C. to 400° C., from 50° C. to 350° C., from 50° C. to 300° C., from 50° C. to 250° C., from 50° C. to 200° C., from 60° C. to 400° C., from 60° C. to 350° C., from 60° C. to 300° C., from 60° C. to 250° C., or from 60° C. to 200° C. Without intending to be bound by any particular theory, it is believed that a reduced reactor temperature, such as less than or equal to 400° C., less than or equal to 350° C., less than or equal to 300° C., or less than or equal to 250° C., can reduce the formation of undesired side products during the contacting. Further, the reduced operational temperature of the reactor can reduce the energy required for the process, which can also reduce the economic cost of operating.

In embodiments, the contacting causes at least a portion of the unsaturated polyethylene to undergo catalytic reactions to produce an effluent. In embodiments, the effluent can comprise hydrocarbons having an average molecular weight of from 40 g/mol to 1000 g/mol. In embodiments, the effluent can comprise at least the alkene product of chemical formula CₘH₂ₘ. In embodiments, the alkene product is a compound of chemical formula CₘH₂ₘ, where m is an integer from 3 to 20. For instance, the alkene product can be a compound of chemical formula CₘH₂ₘ, where m is an integer from 3 to 15, from 3 to 10, from 3 to 8, from 3 to 7, from 3 to 6, from 3 to 5, from 3 to 4, or of 3. In embodiments, the alkene product can comprise propylene, butenes, pentenes, or combinations thereof. In embodiments, the alkene product can be selected from the group consisting of propylene, butenes, pentenes, and combinations thereof. In embodiments, the alkene product can consist essentially of, or consist of, propylene, butenes, pentenes, or combinations thereof. In embodiments, the alkene product can consist essentially of, or consist of propylene.

In embodiments, the effluent can comprise at least 1 wt. %, at least 5 wt. %, at least 10 wt. %, at least 15 wt. %, at least 20 wt. %, at least 25 wt. %, at least 30 wt. %, at least 35 wt. %, at least 40 wt. %, at least 45 wt. %, at least 50 wt. %, at least 55 wt. %, or even at least 60 wt. % of the alkene product.

### EXAMPLES

The various aspects of the present disclosure will be further clarified by the following examples. The examples are illustrative in nature and should not be understood to limit the subject matter of the present disclosure. The materials used in the Examples are provided below in Table 1. In Examples 1-7, catalysts according to the present disclosure were prepared.

TABLE 1

| | Chemical Description, Chemical formula, | |
|---|---|---|
| Product Name | or Structure | Source |
| Methyltrioxorhenium | CH₃ReO₃ (MTO), solid crystal, Re | Sigma- |
| | concentration 71.0-76.0% | Aldrich |
| Ammonium | H₄NO₄Re, solid crystal, ≥99% purity | Sigma- |
| perrhenate | | Aldrich |
| gamma-Alumina | γ-Al₂O₃, surface area 186 m²g⁻¹, pore | Strem |
| | volume 0.50 cm³g⁻¹, 97% purity | Chemicals, |
| | | Inc. |
| Amorphous alumina | a-Al₂O₃, mesoporous powder, pore size 3.8 nm | Sigma- |
| | | Aldrich |
| Carbon | CCl₄, anhydrous, ≥99.5% purity, solvent | Sigma- |
| tetrachloride | | Aldrich |
| trimethylaluminum | Me₃Al, 2M solution in toluene | Sigma- |
| | | Aldrich |
| Carbon disulfide | CS₂, ≥99.9% purity, solvent | Fisher |
| Chloroform | CHCl₃, HPLC grade, OmniSolv ®, CX1054-6 | EMD |
| | | Millipore |
| | | Corp. |
| Xylene | C₈H₁₀, ACS | MP |
| Methanol | CH₃OH, Certified ACS, | Fischer |
| | | Chemical |
| Ethylene | C₂H₄, Ultra high purity (UHP), gas UHP, gas, | Airgas |
| compressed | purified with an oxygen/moisture trap (Supelco) | |
| Oxygen compressed | O₂, UHP, gas | Airgas |
| Argon compressed | Ar, UHP, gas | Airgas |
| Nitrogen compressed | N₂, UHP, gas | Airgas |
| 1-octadecene | C₁₈H₃₆, ≥95% purity, solvent, purified by | Sigma- |
| | standard freeze-pump-thaw degassing method | Aldrich |
| Unsaturated | Mₙ = 1300 g/mol | Dow |
| polyethylene | M_{w} = 14000 g/mol | Chemical |
| Olefin-Terminated | Mₙ = 1000 g/mol | Ex. 8 |
| Polyethylene | | |
| Catalyst | CH₃ReO₃/Cl—Al2O₃, 4 wt. % MTO, 4 wt. % Cl | Ex. 1 |
| Catalyst | Cl—Al₂O₃, 4 wt. % Cl | Ex. 1 |
| Catalyst | Re₂O₇/γ-Al₂O₃, 10 wt. % Re | Ex. 2 |
| Catalyst | Ru[PPh₃]₃[CO][Cl]H | Ex. 3 |
| Catalyst | Re/Al₂O₃, 10 wt. % Re | Ex. 4 |
| Catalyst | CH₃ReO₃/Cl—Al2O₃, 7 wt. % MTO, 4 wt. % Cl | Ex. 5 |
| Catalyst | PtRe/SiO₂, 5 wt. % Pt, 5 wt. % Re | Ex. 6 |
| Catalyst | [^{tBu}POCOP]Ir[C₂H₄] | Ex. 7 |
| Catalyst | 4% CH₃ReO₃/a-Al2O₃ | Comp. Ex. A |
| Catalyst | bis[1-[2,6-diethylphenyl]-3,5,5-trimethyl-3- | Strem |
| | phenylpyrrolidin-2-ylidene][3-phenyl-1H-inden-1- | Chemicals, |
| | ylidene]Ru[II]Cl₂, “UltraCat” | Inc. |
| Catalyst | Acetonitrile(cyclopentadienyl)[2-(di-i-propylphosphino)- | Strem |
| | 4-(t-butyl)-1-methyl-1H-imidazole]ruthenium(II) | Chemicals, |
| | hexafluorophosphate, “Alkene Zipper” | Inc. |
| Catalyst | di-μ-bromo-bis(tris(tert-butylphosphino))dipalladium(I), | Strem |
| | “[Pd^{I}[μ-Br]^{t}Bu₃P]₂” | Chemicals, |
| | | Inc. |

### Example 1. Preparation of 4 wt. % CH₃ReO₃/Cl—Al₂O₃ Catalyst Composition

The catalyst composition of Example 1, 4 wt. % CH₃ReO₃/Cl—Al₂O₃, was synthesized using the following procedure: γ-Al₂O₃ (Strem Chemicals, Inc.) was calcined at 550° C. in air for 4 hours (h), followed by evacuation at 450° C. under dynamic vacuum (10⁻⁴ Torr) overnight. This partially dehydrated and dehydroxylated alumina was chlorinated in a stream of CCl₄-saturated Ar (Airgas, UHP, 10 mL/min) in a fixed bed reactor at 300° C. for 1 h. CCl₄ was distilled prior to use. The resulting Cl—Al₂O₃ was evacuated at 450° C. overnight and modified with CH₃ReO₃ (MTO, Sigma-Aldrich) by vacuum sublimation (ca. 10⁻⁴ Torr) at room temperature to obtain a material containing 4 wt. % MTO and 4 wt. % Cl based on the total weight of the material. Periodically, the solid was shaken vigorously to promote uniform deposition of MTO. After grafting the MTO on the Cl—Al₂O₃, the catalyst was evacuated 30 min at room temperature to remove physisorbed material and the catalyst was stored in a N₂-filled glovebox to prevent deactivation in air.

### Example 2. Preparation of Re₂O₇/γ-Al₂O₃ Catalyst Composition

The catalyst composition of Example 5, Re₂O₇/γ-Al₂O₃, was synthesized using the following procedure: Re₂O₇/γ-Al₂O₃ was prepared by incipient wetness impregnation of γ-Al₂O₃ (Strem Chemicals, Inc.) with ammonium perrhenate to obtain a material containing 10 wt. % Re. Prior to impregnation, γ-Al₂O₃ was calcined at 550° C. for 4 h within 2 h. After impregnation, the dried material was activated by calcination in oxygen at 650° C. at 5° C./min for 8 h. The calcined catalyst was stored in a N₂-filled glovebox until use to avoid deactivation in air.

### Comparative Example A. Preparation of 4 wt. % CH₃ReO₃/Cl—Al₂O₃Catalyst Composition

The catalyst composition of Comparative Example A, 4 wt. % CH₃ReO₃/α-Al₂O₃, was synthesized using the following procedure: α-Al₂O₃ (Sigma Aldrich) was calcined at 550° C. in air for 4 hours (h), followed by evacuation at 450° C. under dynamic vacuum (10⁻⁴ Torr) overnight. This dehydrated α-Al₂O₃ was modified with CH₃ReO₃ (MTO, Sigma-Aldrich) by vacuum sublimation (ca. 10⁻⁴ Torr) at room temperature to obtain a material containing 4 wt. % MTO based on the total weight of the material. Periodically, the solid was shaken vigorously to promote uniform deposition of MTO. After grafting the MTO on the α-Al₂O₃, the catalyst was evacuated 30 min at room temperature to remove physisorbed material and the catalyst was stored in a N₂-filled glovebox to prevent deactivation in air.

### Comparative Example B. Preparation of γ-Al₂O₃ Catalyst Composition

The catalyst composition of Comparative Example B was prepared as follows: γ-Al₂O₃ (Strem Chemicals, Inc.) was calcined at 550° C. in air for 4 hours (h), followed by evacuation at 450° C. under dynamic vacuum (10⁻⁴ Torr) overnight.

### Example 3. Preparation of Ru[PPh₃]₃[CO][Cl]H Catalyst Composition

The catalyst composition of Example 3, Ru[PPh₃]₃[CO][Cl]H, was synthesized according to Prasanna, N.; Synthesis, Spectral and Electrochemical Studies of Ruthenium(II)/(III) Complexes of Alicyclic B-Ketamines, Indian J. Chem. 2001, 40, 426-429.

### Example 4. Preparation of Re/Al₂O₃Catalyst Composition

The catalyst composition of Example 4, 10 wt. % Re/Al₂O₃ was synthesized according to the following procedure: 1024 mg of γ-Al₂O₃ was placed into a round-bottom flask with a stir bar and heated to 110° C. using a hot oil bath and dried for 24 hours. 163 mg of ammonium perrhenate was added to 5 mL of deionized water to form a mixture and the mixture was added to the dried γ-Al₂O₃. The slurry was stirred overnight at 80° C. The resulting solids were calcined within a furnace under a flow of dry air for 8 hours. The furnace ramping rate was 5° C./min up to 650° C. The catalyst was cooled to room temperature under a flow of helium and transferred to an Ar-filled glovebox for storage to obtain a catalyst composition comprising 10 wt. % Re based on the total weight of the catalyst composition.

### Example 5. Preparation of 7 wt. % CH₃ReO₃/Cl—Al₂O₃ Catalyst Composition

The catalyst composition of Example 5, 7 wt. % CH₃ReO₃/Cl—Al₂O₃, was synthesized using the following procedure: MTO was sublimed over 5 hours onto 816 mg of Al₂O₃/Cl (4 wt %) that was held at −78° C. using a liquid N₂ trap. After complete sublimation of MTO, the mixture was stirred for 0.5 hours and warmed to room temperature under static vacuum. The solid was then left under dynamic vacuum for another 0.5 hours. The material was inertly transferred to an Ar-filled glovebox for storage.

### Example 6. Preparation of PtRe/SiO₂ Catalyst Composition

The catalyst composition of Example 6, PtRe/SiO₂, was prepared using the following procedure: PtRe/SiO₂ was prepared by incipient wetness impregnation of silica powder with ammonium perrhenate to obtain a material containing 1-5 wt % Re. After impregnation, the material was calcined at 500° C. Pt was deposited on the material by incipient wetness impregnation in toluene with platinum acetylacetonate to obtain a material containing 1-5 wt % Pt. The resulting solid was dried in air at 120° C. for 4 h after which the temperature was increased to 210° C. for 4 h. The material was reduced in H₂ at 150° C. for 1 h. The reduced catalyst was stored in a N₂ atmosphere until use to avoid re-oxidation in air. The PtRe/SiO₂ catalyst was calcined at 500° C. for 4 h followed by reduction with H₂ at 280° C. for 2 h. The heating rate is 2° C./min. After reduction, the catalyst was evacuated 30 min at room temperature to remove physisorbed H₂ and stored in N₂-filled glovebox to prevent deactivation in air.

### Example 7. Preparation of [^{tBu}POCOP]Ir[C₂H₄] Catalyst Composition

The catalyst composition of Example 7, [^{tBu}POCOP]Ir[C₂H₄], was prepared according to “Catalytic Alkane Metathesis by Tandem Alkane Dehydrogenation-Olefin Metathesis” Science 2006, 312, 257-261. [C₆H₃-2,6-[OP(t-Bu)₂]₂]Ir[H][Cl] and NaO-t-Bu were weighed into an oven-dried Schlenk flask in a molar ratio of 1 to 1.2, respectively. The solids were then put under a flow of argon. 40 mL of toluene was added to the flask via syringe, and the resulting suspension was stirred for 10 min at room temperature. Ethylene was bubbled through the solution for 1-2 hours. The solution was cannula-filtered through a pad of Celite, volatiles were evaporated under vacuum, and the resulting red solid was dried under vacuum overnight to afford the product in 60% yield.

### Example 8. Preparation of Olefin-Terminated Polyethylene

In Example 8, olefin-terminated polyethylene was prepared as follows: A 300 ml Parr HP 5500 Compact reactor equipped with an overhead stirrer was degassed with Ar. Afterwards, 100 ml of toluene was transferred into the reactor, and heated to 60° C. Inside an Ar-filled glovebox, 2.8 mg (5 μmol) of {κ2-P, O-2-[Di(2-methoxyphenyl)phosphino]benzenesulphonato}nickel(II)-methyl pyridine was weighed into a dry 7-mL glass vial and dissolved in 1 mL toluene. The catalytic solution was transferred, inertly, into the Parr reactor using an air-tight syringe. The reactor was charged with ethylene (35 bar), and the polymerization reaction was carried out for 60 minutes. The reactor is then vented and cooled to room temperature. The resulting polymer solution was precipitated in excess methanol (500 mL) and collected via vacuum filtration. The material was dried in vacuo at 43° C. Because the product polymer contains a single unsaturated bond per polymer chain, normalizing the —CH₂— protons by the number of olefinic protons (terminal and internal) provides an estimate of polymer molecular weight (M_{N}). ¹H NMR (toluene-d₈), alkyl-CH₂: 0.92-2.00 (421.5H, s), terminal-α: 4.97 (2.01, dd), internal: 5.44 (2.85, m), terminal-β: 5.8 (1.00, m).

In examples 9-11, catalytic processes according to the present disclosure were carried out in a batch reactor. Hydrocarbons in the gas fraction product (C₁-C₆) were analyzed quantitatively on a Shimadzu GC-2010 gas chromatograph equipped with a capillary column (Supelco Alumina Sulfate plot, 30 m×0.32 mm) and a flame ionization detector (FID). The signal coefficient is dependent on the carbon number for each hydrocarbon species. The injector and detector temperatures were 200° C. The temperature ramp program was as follows: 90° C. (hold 3 min), ramp 10° C./min to 150° C. (hold 20 min). Helium was used as carrier gas. H₂, C₂H₄, and C₂H₆ were quantified on a Shimadzu GC-8AIT gas chromatograph equipped with a packed column (ShinCarbon ST 80/100, 2 m×2 mm) and a thermal conductivity detector (TCD). The linear response of the TCD signal to the injected volumes of H₂, C₂H₄, and C₂H₆ was confirmed using standard gas mixtures. The response factors were obtained as the slopes of fitted lines. The column, injector and detector temperatures were 130° C. The TCD current was 70 mA and the carrier gas pressure was 300 kPa (N₂). Liquid phase products (>C₅) were analyzed on an Agilent 6890N Network Gas Chromatograph equipped with a DB-5 column and an FID detector. ¹H NMR spectra were recorded in 1,1,2,2-tetrachloroethane-d₂ at 600 MHz on a Varian Unity Inova AS600 spectrometer, and were analyzed using MestReNova (v11.0.1, Mestrelab Research S. L.). Chemical shifts (δ, ppm) were calibrated using the residual proton signals of the solvent and referenced to tetramethylsilane (TMS).

### Example 9. Catalytic Conversion of Unsaturated Polyethylene in a Batch Reactor

In Example 9, unsaturated polyethylene (Mₙ=1300 g/mol) was reacted with ethylene over one of catalyst compositions: Comparative Ex. A, or Ex. 1 in a 10 mL batch reactor (Parr reactor, Series 2550), according to Table 2. In an N₂-filled glovebox, the catalyst composition, the unsaturated polyethylene, and the unsaturated polyethylene were loaded into the reactor equipped with a pressure gauge and type K thermocouple. Ethylene (99.999%, Airgas) was passed through a moisture/oxygen trap (Supelco) before use. Gas lines were purged of residual air for three 5-min cycles before ethylene was introduced into the reactor. Reactor heating was initiated, and reaction time was tracked after reaching a desired temperature of 130° C. After a reaction time of 3 hours, the reactor was cooled in flowing air. Aliquots of gas from the reactor headspace were taken for GC analysis before venting the rest of the headspace in a fume hood. The remaining solid and liquid was transferred onto a fine glass filter (4.0-5.5 μm) and filtered to remove insoluble material by washing with hot (50° C.) CHCl₃. Soluble hydrocarbons were recovered by evaporating the solvent under reduced pressure (0.1 Torr). The insoluble material, including the catalyst and hydrocarbons insoluble in hot CHCl₃, was recovered from the filter. The solids were analyzed by GPC. The results of the products formed in Example 9 are shown in Table 3.

TABLE 2

| | Unsaturated | Ethylene | | Catalyst | Reaction | Reaction |
|---|---|---|---|---|---|---|
| | PE mass | mass | Catalyst | mass | temperature | time |
| Example | (mg) | (mg) | Composition | (mg) | (° C.) | (hours) |
| Comp. | 124 | 240 | Comp. | 200 | 130 | 3 |
| Ex. 9A | | | Ex. A | | | |
| Comp. | — | 238 | Ex. 1 | 199 | 130 | 3 |
| Ex. 9B | | | | | | |
| Ex. 9-1 | 123 | 245 | Ex. 1 | 200 | 130 | 3 |

TABLE 3

| | | | Mₙ of | M_{w} of |
|---|---|---|---|---|
| | Propylene | 2-butene | product | product |
| Tested | produced | produced | distribution | distribution |
| Property | (mg) | (mg) | (g/mol) | (g/mol) |
| Comp. Ex. 9A | 5 | 0.5 | 1270 | 2370 |
| Comp. Ex. 9B | 15 | 1.5 | No data | No data |
| Ex. 9-1 | 67 | 6 | 820 | 1440 |

### Example 10. Catalytic Conversion of Unsaturated Polyethylene in a Batch Reactor at Varied Reaction Times to Form Alkenes

In Example 10, unsaturated polyethylene (Mₙ=1300 g/mol) was reacted with ethylene over one of catalyst compositions: Ex. 1, or Ex. 2 in a batch reactor (Ex. 10-1 and 10-2: 10 mL Parr reactor, Series 2550; Ex. 10-3, Comp. Ex. 10A and 10B: 25 mL Parr reactor, Series 4590), according to Table 4. In an N₂-filled glovebox, the catalyst composition and the unsaturated polyethylene, were loaded into the reactor equipped with a pressure gauge and type K thermocouple. Ethylene (99.999%, Airgas) was passed through a moisture/oxygen trap (Supelco) before use. Gas lines were purged of residual air for three 5-min cycles before ethylene was introduced into the reactor. Reactor heating was initiated. The reaction time was tracked after reaching a designated temperature according to Table 4. After the designated reaction time, the reactor was cooled in flowing air. Aliquots of gas from the reactor headspace were taken for GC analysis before venting the rest of the headspace in a fume hood. The remaining solid and liquid was transferred onto a fine glass filter (4.0-5.5 μm) and filtered to remove insoluble material by washing with hot (50° C.) CHCl₃. Soluble hydrocarbons were recovered by evaporating the solvent under reduced pressure (0.1 Torr). The insoluble material, including the catalyst and hydrocarbons insoluble in hot CHCl₃, was recovered from the filter. The solids were analyzed by GPC. The results of the products formed in Example 10 are shown in Table 5.

TABLE 4

| | Unsaturated | Ethylene | | Catalyst | Reaction | Reaction |
|---|---|---|---|---|---|---|
| | PE mass | mass | Catalyst | mass | temperature | time |
| Example | (mg) | (mg) | Composition | (mg) | (° C.) | (hours) |
| Ex. 10-1 | 119 | 236 | Ex. 1 | 199 | 130 | 1 |
| Ex. 10-2 | 130 | 245 | Ex. 1 | 213 | 130 | 6 |
| Ex. 10-3 | 240 | 1130 | Ex. 2 | 400 | 200 | 24 |
| Comp. | — | 1299 | Ex. 2 | 200 | 200 | 24 |
| Ex. 10A | | | | | | |
| Comp. | 120 | — | Ex. 2 | 200 | 200 | 24 |
| Ex. 10B | | | | | | |

TABLE 5

| | Propylene | 2-butene | Mₙ of product | M_{w} of product |
|---|---|---|---|---|
| Tested | produced | produced | distribution | distribution |
| Property | (mg) | (mg) | (g/mol) | (g/mol) |
| Ex. 10-1 | 60 | 7 | No data | No data |
| Ex. 10-2 | 64 | 7 | No data | No data |
| Ex. 10-3 | 16 | 57 | 1906 | 7337 |
| Comp. Ex. 10A | 3 | 14 | No data | No data |
| Comp. Ex. 10A | 0 | 0 | 2417 | 21269 |

### Example 11. Catalytic Conversion of 1-Octadecene Over Catalyst Composition Example 1 in a Batch Reactor

In Example 11, 1-octadecene was reacted with ethylene over the catalyst composition of Ex. 1, and in a batch reactor (Parr reactor, Series 2550) according to Table 6. 1-octadecene was degassed using three freeze-pump-thaw cycles and then transferred into a N₂-filled glovebox. The liquid was then dried with molecular sieves (3 Å). In an N₂-filled glovebox, the catalyst composition of Ex. 1 and 1-octadecene were loaded into a Parr reactor (10 mL, Series 2550) equipped with a pressure gauge and type K thermocouple. Ethylene (99.999%, Airgas) was passed through a moisture/oxygen trap (Supelco) before use. Gas lines were purged of residual air for three cycles before ethylene was introduced into the reactor. Heating to the designated temperature after pressurization with ethylene. After the designated reaction time, the reactor was cooled in flowing air. Aliquots of gas and liquids from the reactor headspace were taken for gas chromatography analysis with a flame ionization detector (GC-FID). The remaining solid and liquid was transferred onto a fine glass filter (4.0-5.5 μm) and filtered to remove insoluble material, such as coke, by washing with 5 mL CS₂ solution. After filtration, the filtrate solution was analyzed by GC-FID. 1H NMR spectra of the reaction products was measured. The peaks characteristic of internal olefins and terminal olefins were integrated. The ratio of the internal olefins to terminal olefins was calculated. The results are shown in

### Table 7.

TABLE 6

| | 1- | | | | | | | |
|---|---|---|---|---|---|---|---|---|
| | octadecene | Ethylene | Ethylene | N₂ | | Catalyst | Reaction | Reaction |
| | mass | mass | Pressure | mass | Catalyst | mass | temperature | time |
| Example | (mg) | (mg) | (psig) | (mg) | Composition | (mg) | (° C.) | (hours) |
| Ex. 11-1 | 400 | 201 | 259 | | Ex. 1 | 144 | 100 | 6 |
| Ex. 11-2 | 400 | 204 | 263 | | Ex. 1 | 204 | 100 | 3 |
| Ex. 11-3 | 200 | 505 | 649 | | Ex. 1 | 100 | 100 | 1 |
| Ex. 11-4 | 202 | 38 | 49 | | Ex. 1 | 101 | 100 | 1 |
| Ex. 11-5 | 200 | 89 | 115 | | Ex. 1 | 96 | 100 | 1 |
| Ex. 11-6 | 394 | 1299 | 656 | | Ex. 2 | 200 | 200 | 24 |
| Comp. | 200 | — | — | 85 | Ex. 1 | 96 | 100 | 1 |
| Ex. 11A | | | | | | | | |
| Comp. | 394 | — | — | — | Comp. | 202 | 200 | 24 |
| Ex. 11B | | | | | Ex. B | | | |

TABLE 7

| | | | CS₂- | CS₂- | | | |
|---|---|---|---|---|---|---|---|
| | | | soluble | insoluble | Mₙ of | M_{w} of | Internal |
| | Propylene | 2-butene | olefin | olefin | product | product | olefin:external |
| Tested | produced | produced | products | products | distribution | distribution | olefin |
| Property | (mg) | (mg) | (mg) | (mg) | (g/mol) | (g/mol) | ratio |
| Ex. 11-1 | 33 | 3.6 | No data | No data | No data | No data | No data |
| Ex. 11-2 | 67 | 8 | No data | No data | No data | No data | No data |
| Ex. 11-3 | 61 | 3.7 | 116 | 23 | 68 | 95 | No data |
| Ex. 11-4 | 3 | 1.2 | 196 | 8.8 | 210 | 270 | 18 |
| Ex. 11-5 | 28 | 0.8 | 182 | 16 | 110 | 210 | 8 |
| Ex. 11-6 | 40 | 87 | No data | No data | No data | No data | No data |
| Comp. | 0 | 0 | 198 | 7 | 410 | 430 | 440 |
| Ex. 11A | | | | | | | |
| Comp. | 0 | 0 | 392 | 2 | 252 | 252 | 18 |
| Ex. 11B | | | | | | | |

In Examples 12, a stirred tank reactor was used, as shown in FIG. 1. A 20 mL glass reaction sleeve (ID=19.56 mm, OD=22.15 mm) was placed into a 40 mL stainless-steel stirred-tank reactor (ID=22.16 mm, OD=40 mm), and the reactor was housed within an aluminum heating jacket. The temperature of the heating jacket was controlled by a hotplate and thermocouple (IKA C-MAG HS7 digital). The reactor has two inlet ports, one for liquid substrates and a second for gaseous substrates. Liquid substrates were delivered into the setup using a Hamilton gas-tight syringe (5 mL) and a Kd Scientific Legato 100 Syringe Pump. Gaseous substrates were supplied from a pressurized tank whose flow was set by an Alicat mass flow controller (MCS series). The outlet stream led to an Equilibar backpressure regulator which was used to control the reaction pressure. Attached downstream from the regulator was an Agilent 6850 gas chromatograph (GC). The GC was equipped with a 6-port VICI-Valco gas-sampling valve. A continuous flow of ethylene was used as an internal standard to quantify olefin formation rates. Stainless-steel tubing and fittings were purchased from McMaster-Carr and Swagelok. The GC was equipped with an FID and a Petrocol DH Capillary GC Column (100 mm×0.25 mm×0.5 μm film thickness). The column was held at 45 PSI and the gaseous sample was split 50:1. The column conditions and product elution times are shown in Table 8 and Table 9, respectively.

TABLE 8

| Oven Ramp | ° C./min | Next ° C. | Hold min |
|---|---|---|---|
| Initial | | 50 | 8 |
| Ramp1 | 15 | 100 | 1 |
| Ramp2 | 15 | 150 | 15.00 |

TABLE 9

| Hydrocarbon Species | Elution Time min |
|---|---|
| Ethylene | 8.2 |
| Propylene | 8.3 |
| Butenes | 8.8-9.0 |
| Pentenes | 10.1-9.5 |
| Hexenes | 12.2-14.1 |
| Heptenes | 15.1-17.0 |
| Octenes | 19.0-20.1 |
| Nonenes | 22.0-24.1 |
| Decenes | 25.4-26.3 |

In Example 12, the olefin terminated polyethylene, as prepared in Example 8 was reacted with ethylene and catalyst compositions described herein in the stirred-tank reactor using various reaction conditions. The maximum propylene formation rate detected while catalyst is on-stream (R_{C3, max}), in millimoles per hour (mmol h⁻¹), was measured. The maximum propylene selectivity (S_{C3, max}) and butylenes selectivity (S_{C4, max}) while the catalyst was on-stream was measured. The formation rate of propylene and butylenes are normalized by the cumulative olefin formation rate for a given reaction time. The average of the selectivity of propylene (S_{C3,avg}) and butylenes (S_{C4,avg}) were evaluated at each sampling point during the course of the continuous reaction for each species. The polyethylene conversion, in weight percent, was also estimated by calculating the mass of polyethylene consumed per olefin produced, according to equation 1:

$$\frac{\sum ( \frac{i - 2}{i} {n}_{Ci} M {W}_{i} ) * 100}{{m}_{PE , o}} \qquad ( 1 )$$

where i represents the number of carbon units within the olefin, MWᵢ represents the molecular weight of species “i”, n_{ci} is the moles of species “i” formed during the experiment, and m_{PE,o} is the initial loading of polyethylene, assuming each molecule of olefin formed contains two carbons from ethylene. The results are summarized in Table 10.

### Example 12. Catalytic Conversion of Olefin-Terminated Polyethylene in a Stirred-Tank Reactor to Form Alkenes

Example 12-1 was carried out as follows: within an Ar-filled glovebox, 157 mg of olefin-terminated polyethylene was loaded into a stirred-tank reactor. The reactor was removed from the glovebox, placed within an aluminum heating jacket, and connected to an ethylene delivery source. The Ar-atmosphere within the reactor was evacuated using a continuous flow of ethylene fed at 5 mL/min for at least 15 minutes. A catalyst solution was prepared within an Ar-filled glovebox by mixing 5 mL of toluene with 53.0 mg of Ultracat in a 20 mL scintillation vial. The solution was stirred for 3 to 5 minutes and then transferred to a 5 ml syringe capped with a needle and septum for inert delivery via syringe pump. For 9 hours, the catalyst solution (0.01 mL/min) was co-fed with ethylene gas (2.2 mL/min) into the reactor which was heated to and held at 85° C. and 5.4 atm. To monitor reaction progression, a sample of the gaseous effluent (0.25 mL) was analyzed via GC every 34.2 minutes for the 9-hour duration of the reaction.

Example 12-2, was carried out according to Example 12-1, but 100 mg of Ex. 6 was loaded into the stirred-tank reactor with the PE before heating, and the reaction temperature was 100° C. Additionally, Ex. 12-2 used 250 mg of the PE instead of 157 mg, and used 51.0 mg of UltraCat instead of 53 mg.

Example 12-3 was carried out according to Example 12-2, but 19.4 mg of Ex. 3 was loaded into the stirred-tank reactor with the PE before heating, instead of Ex. 6.

Example 12-4 was carried out as follows: within an Ar-filled glovebox, 250 mg of Olefin-Terminated Polyethylene was loaded into a stirred-tank reactor. The reactor was removed from the glovebox, placed within an aluminum heating jacket, and connected to an ethylene delivery source. The Ar-atmosphere within the reactor was evacuated using a continuous flow of ethylene fed at 5 mL/min for at least 15 minutes. A catalyst solution was prepared within an Ar-filled glovebox by mixing 5 mL of toluene with 50.0 mg of Ultracat and 20 mg of Ex. 3 in a 20 mL scintillation vial. The solution was stirred for 3 to 5 minutes and then transferred to a 5 mL syringe capped with a needle and septum for inert delivery via syringe pump. For 8 hours, the catalyst solution (0.01 mL/min) was co-fed with ethylene gas (2.2 mL/min) into the reactor which was heated to and held at 100° C. and 5.4 atm. To monitor reaction progression, a sample of the gaseous effluent (0.25 mL) was analyzed via GC every 34.2 minutes for the 8-hour duration of the reaction.

Example 12-5 was carried out according to Example 12-4, but the reaction pressure was 1 atm instead of 5.4 atm.

Example 12-6 was carried out according to Example 12-4, but the reaction temperature was 70° C., the pressure was 1.0 atm, and time was 9 hours. Additionally, after the 9-hour reaction, the mixture was cooled and left to sit for two days. A second catalyst feed of identical concentration was prepared in an Ar-filled glovebox. Subsequently, the catalyst solution (0.01 mL/min) was co-fed with ethylene gas (2.2 mL/min) into the reactor which was heated to and held at 70° C. and 1 atm for an additional 8-hours. To monitor reaction progression, a sample of the gaseous effluent (0.25 mL) was analyzed via GC every 34.2 minutes for the 8-hour duration of the reaction.

Example 12-7 was carried out according to Example 12-5, but the mass of Ex. 3 used to prepare the catalyst solution was 103 mg, instead of 20 mg.

Example 12-8 was carried out as follows: within an Ar-filled glovebox, 253 mg of the olefin-terminated polyethylene of Ex. 8 was loaded into a stirred-tank reactor. Next, the reactor was removed from the glovebox, placed within an aluminum heating jacket, and connected to an ethylene delivery source. The Ar-atmosphere within the reactor was evacuated using a continuous flow of ethylene fed at 5 mL/min for at least 15 minutes. A catalyst solution was prepared within an Ar-filled glovebox by mixing 5 mL of toluene, 50.0 mg of UltraCat, and 42.0 mg of [Pd^{I}[μ-Br]^{t}Bu₃P]₂ (Strem Chemicals, Inc.) in a 20 mL scintillation vial. The solution was stirred for 3 to 5 minutes and then transferred to a 5 mL syringe capped with a needle and septum for inert delivery via syringe pump. For 9 hours, the catalyst solution (0.01 mL/min) was co-fed with ethylene gas (10.1 mL/min) into the reactor which was heated to and held at 70° C. and 1 atm. To monitor reaction progression, a sample of the gaseous effluent (0.25 mL) was analyzed via GC every 34.2 minutes for the 9-hour duration of the reaction.

Example 12-9 was carried out according to Example 12-8, but the catalyst solution was prepared using 61 mg of Alkene Zipper (Strem Chemicals, Inc.), instead of [Pd^{I}[μ-Br]^{t}Bu₃P]₂.

Example 12-10 was carried out as follows: within an Ar-filled glovebox, 253 mg of the olefin-terminated polyethylene of Ex. 8 and 202 mg of Ex. 5 were loaded into a stirred-tank reactor. Next, the reactor was removed from the glovebox, placed within an aluminum heating jacket, and connected to an ethylene delivery source. The Ar-atmosphere within the reactor was evacuated using a continuous flow of ethylene fed at 5 mL/min for at least 15 minutes. For 20 hours, ethylene gas (10.1 mL/min) was continuously flown into the reactor which was heated to and held at 100° C. and 1 atm for 20 hours. To monitor reaction progression, a sample of the gaseous effluent (0.25 mL) was analyzed via GC every 34.2 minutes for the 20-hour duration of the reaction.

Example 12-11 was carried out according to 12-10, but 14.5 mg of [Pd^{I}[μ-Br]^{t}Bu₃P]₂ (Strem) was loaded into the stirred-tank reactor in addition to Ex. 5. The reaction was carried out for 10.5 hours instead of 20 hours.

Example 12-12 was carried out according to 12-11, but 51 mg of Ex. 6 was loaded into the stirred-tank reactor instead of [Pd^{I}[μ-Br]^{t}Bu₃P]₂. 149 mg of UltraCat was used instead of 202 mg. The reaction was carried out for 8 hours instead of 10.5 hours.

Example 12-13 was carried out according to 12-12, but 24.6 mg of Ex. 7 was loaded into the stirred-tank reactor instead of Ex. 6. 156 mg of UltraCat was used instead of 149 mg. The reaction was carried out for 19.5 hours instead of 8 hours.

Example 12-14 was carried out as follows: within an Ar-filled glovebox, 150 mg of the olefin-terminated polyethylene of Ex. 8, 140 mg of Ex. 4, 150 mg of Ex. 6, and 0.075 mL of Me₃Al dissolved in toluene (2M) were loaded into a stirred-tank reactor. Next, the reactor was removed from the glovebox, placed within an aluminum heating jacket, and connected to an ethylene delivery source. The Ar-atmosphere within the reactor was evacuated using a continuous flow of ethylene fed at 5 mL/min for at least 15 minutes. For 10 hours, ethylene gas (2.2 mL/min) was continuously flown into the reactor which was heated to and held at 170° C. and 6.8 atm. To monitor reaction progression, a sample of the gaseous effluent (0.25 mL) was analyzed via GC every 34.2 minutes for the 10-hour duration of the reaction.

Example 12-15 was carried out according to Example 12-14, but 3 mg of Ex. 7 was loaded into the stirred-tank reactor instead of Ex. 6. The reaction temperature was 150° C. instead of 170° C.

TABLE 10

| Example | Ex. 12-1 | Ex. 12-2 | Ex. 12-3 | Ex. 12-4 | Ex. 12-5 | Ex. 12-6 | Ex. 12-7 | Ex. 12-8 |
|---|---|---|---|---|---|---|---|---|
| Product | C3-C4 | C3-C4 | C3-C5 | C3-C6 | C3-C6 | C3-C6 | C3-C5 | C3-C8 |
| Detected | | | | | | | | |
| RC3 max | 0.04 | 0.04 | 0.12 | 0.15 | 0.07 | 0.44 | 0.93 | 0.88 |
| (mmol/h) | | | | | | | | |
| SC3 max | 0.68 | 0.92 | 0.93 | 0.94 | 0.94 | 0.98 | 0.96 | 0.8 |
| Selectivity | 0.55 | 0.74 | 0.78 | 0.9 | 0.9 | 0.93 | 0.94 | 0.78 |
| (SC3avg) | | | | | | | | |
| Selectivity | 0.45 | 0.26 | 0.15 | 0.08 | 0.05 | 0.05 | 0.05 | 0.16 |
| (SC4avg) | | | | | | | | |
| Selectivity | 0 | 0 | 0.07 | 0.02 | 0.05 | 0.02 | 0.01 | 0.04 |
| (SC4+) | | | | | | | | |
| PE | >1.0 | 1 | 2.1 | 1.5 | 4.2 | 4.8 | 19.4 | 29.1 |
| Conversion | | | | | | | | |
| (wt %) | | | | | | | | |

| Example | Ex. 12-9 | Ex. 12-10 | Ex. 12-11 | Ex. 12-12 | Ex. 12-13 | Ex. 12-14 | Ex. 12-15 |
|---|---|---|---|---|---|---|---|
| Product | C3-C7 | C3-C11 | C3-C11 | C3-C8 | C3-C11 | C3-C7 | C3-C7 |
| Detected | | | | | | | |
| RC3 max | 0.256 | 2.78 | 3.14 | 1.21 | 0.85 | 0.03 | 0.22 |
| (mmol/h) | | | | | | | |
| SC3 max | 0.79 | 0.84 | 0.88 | 0.78 | 0.93 | 0.65 | 0.91 |
| Selectivity | 0.52 | 0.8 | 0.73 | 0.7 | 0.78 | 0.51 | 0.52 |
| (SC3avg) | | | | | | | |
| Selectivity | 0.22 | 0.14 | 0.19 | 0.14 | 0.15 | 0.31 | 0.39 |
| (SC4avg) | | | | | | | |
| Selectivity | 0.26 | 0.06 | 0.08 | 0.16 | 0.07 | 0.18 | 0.09 |
| (SC4+) | | | | | | | |
| PE | 1.9 | 38 | 45 | 13 | 35.7 | 1.8 | 4.8 |
| Conversion | | | | | | | |
| (wt %) | | | | | | | |

### Example 13. Catalytic Conversion of Olefin-Terminated Polyethylene in a Stirred-Tank Reactor to Form Alkenes at Low Temperature

Example 13-1 was carried out as follows: within an Ar-filled glovebox, 250 mg of Olefin-Terminated Polyethylene was loaded into a stirred-tank reactor with 2 mL of toluene. Next, the reactor was removed from the glovebox, placed within an aluminum heating jacket, and connected to an ethylene delivery source. The Ar-atmosphere within the reactor was evacuated using a continuous flow of ethylene fed at 5 mL/min for at least 10 minutes. A catalyst solution was prepared within an Ar-filled glovebox by mixing 3 mL of toluene, 50.0 mg of UltraCat, and 9.5 mg of [Pd^{I}[μ-Br]^{t}Bu₃P]₂ in a 20 mL scintillation vial. The solution was stirred for 5 to 10 minutes and then transferred to a 5 mL syringe capped with a needle and septum for inert delivery via syringe pump. For 5 hours, the catalyst solution (0.01 mL/min) was co-fed with ethylene gas (5.0 mL/min) into the reactor which was heated to and held at 35° C. and 2 atm. To monitor reaction progression, a sample of the gaseous effluent (0.25 mL) was analyzed via GC every 10 minutes for the 9-hour duration of the reaction.

Example 13-2 was carried out as follows: within an Ar-filled glovebox, 250 mg of Olefin-Terminated Polyethylene was loaded into a stirred-tank reactor with 1 mL of toluene. Next, the reactor was removed from the glovebox, placed within an aluminum heating jacket, and allowed to heat up to 70° C. for 30 min. Next, the reactor was connected to an ethylene and nitrogen delivery source. The Ar-atmosphere within the reactor was evacuated using a continuous flow of ethylene and nitrogen fed at 5 mL/min each for at least 5 minutes. Two catalyst solutions were prepared within an Ar-filled glovebox by mixing 2 mL of toluene with 30 mg of C₃₁H₃₈Cl₂N₂ORu, and 2 mL of toluene with 16 mg of [Pd^{I}[μ-Br]^{t}Bu₃P]₂ in a 20 mL scintillation vial. The solution was stirred for at least 15 minutes and then transferred to two separate 5 mL syringes capped with a needle and septum for inert delivery via syringe pump. First the [Pd^{I}[μ-Br]^{t}Bu₃P]₂ catalyst solution was fed into the reactor followed by C₃₁H₃₈Cl₂N₂ORu solution for 5 hours (0.01 mL/min), co-fed with ethylene gas (5.0 mL/min) and nitrogen gas (5.0 mL/min) into the reactor which was heated to and held at 35° C. and 1 atm. To monitor reaction progression, a sample of the gaseous effluent (0.25 mL) was analyzed via GC every 10 minutes for the 9-hour duration of the reaction.

TABLE 11

| Example | | Olefins | ^{A}R_{C3, max}/ | | | | | ^{D}PE |
|---|---|---|---|---|---|---|---|---|
| No. | Catalyst(s) | Detected | mmol h⁻¹ | ^{B}S_{C3, max} | ^{C}S_{C3, avg} | ^{C}S_{C4, avg} | ^{C}S_{C4+, avg} | Conversion/wt % |
| Example | Ultracat | C₃-C₄ | 0.4 | 1 | .76 | 0.01 | 0 | 18 |
| 13-1 | 3-Pd | | | | | | | |
| Example | C₃₁H₃₈Cl₂N₂ORu | C₃-C₄ | 1.5 | 0.91 | .78 | 0.21 | 0 | 38 |
| 13-2 | 3-Pd | | | | | | | |

^{A}Maximum propylene formation rate detected while catalyst is on-stream.
^{B}This value describes the maximum selectivity that was evaluated while the catalyst was on-stream. The formation rate propylene is normalized by the cumulative olefin formation rate for a given reaction time.
^{C}This value is an average of the selectivity evaluated at each sampling point during the course of the continuous reaction for a given species.
^{D}PE conversion is estimated by calculating the mass of PE consumed per olefin produced (see Equation 1).

### Example 14. Catalytic Conversion of Unsaturated Polyethylene in a Batch Reactor Using Propylene as the Alkene Reactant

In Example 14, unsaturated polyethylene (Mₙ=1300 g/mol, Dispersity=11) was reacted with varying amounts of propylene over the catalyst composition of Ex. 1 in a 10 mL batch reactor (Parr reactor, Series 2550) for varying amounts of time, according to Table 2. In an N₂-filled glovebox, the catalyst composition and the unsaturated polyethylene were loaded into the reactor equipped with a pressure gauge and type K thermocouple. Propylene (99.8%, Praxair) was passed through a moisture/oxygen trap (Supelco) before use. Gas lines were extensively evacuated to remove residual air before propylene was introduced into the reactor. Reactor heating was initiated, and reaction time was tracked after reaching a desired temperature of 130° C. After a reaction time of 1 or 5 hours, the reactor was cooled in flowing air. Aliquots of gas from the reactor headspace were taken for GC analysis before venting the rest of the headspace in a fume hood. The remaining solid was transferred into a 20 mL vial containing xylene at 110° C. to dissolve products from catalyst. The xylene solution was mixed with 180 mL of room temperature methanol to precipitate the extracted products which were separated from methanol via vacuum filtration. Comparative Example 14-4 was prepared similarly to Example 14-1, but saturated polyethylene was used instead of unsaturated polyethylene. The products were analyzed by GPC. The results of the products formed in Example 14 are shown in Table 33.

TABLE 111

| | | Propylene | | Catalyst | Reaction | Reaction |
|---|---|---|---|---|---|---|
| | PE mass | mass | Catalyst | mass | temperature | time |
| Example | (mg) | (mg) | Composition | (mg) | (° C.) | (hours) |
| 14-1 | 123 | 640 | Ex. 1 | 204 | 130 | 1 |
| 14-2 | 120 | 570 | Ex. 1 | 200 | 130 | 5 |
| 14-3 | 123 | 110 | Ex. 1 | 201 | 130 | 5 |
| Comp. | 122 | 610 | Ex. 1 | 206 | 130 | 1 |
| Ex. 14-4 | (Saturated | | | | | |
| | PE) | | | | | |

TABLE 112

| | | | Mₙ of | M_{w} of |
|---|---|---|---|---|
| | Ethylene | 2-butene | product | product |
| | produced | produced | distribution | distribution |
| Example | (mg) | (mg) | (g/mol) | (g/mol) |
| 14-1 | 22 | 7.0 | 1145 | 6618 |
| 14-2 | 47 | 6.5 | 1174 | 6771 |
| 14-3 | 5.5 | 6.0 | 1534 | 9036 |
| Comp. Ex. 14-4 | 4.5 | 5.6 | 1685 | 3854 |

### Example 15. Catalytic Conversion of Unsaturated Polyethylene in a Batch Reactor Over Catalyst Composition Example 1 in a Batch Reactor

In Example 15, unsaturated polyethylene (Mₙ=1300 g/mol, Dispersity=11) was reacted with ethylene over a mixture of MTO/Cl—Al₂O₃ and Cl—Al₂O₃ in a 10 mL batch reactor (Parr reactor, Series 2550), according to Table 14. In an N₂-filled glovebox, the catalyst composition mixture, the unsaturated polyethylene, and the unsaturated polyethylene were loaded into the reactor equipped with a pressure gauge and type K thermocouple. Ethylene (99.999%, Airgas) was passed through a moisture/oxygen trap (Supelco) before use. Gas lines were purged of residual air for three 5-min cycles before ethylene was introduced into the reactor. Reactor heating was initiated, and reaction time was tracked after reaching a desired temperature of 130° C. After a reaction time of 3 hours, the reactor was cooled in flowing air. Aliquots of gas from the reactor headspace were taken for GC analysis before venting the rest of the headspace in a fume hood. The remaining solid was transferred into a 20 mL vial containing xylene at 110° C. to dissolve products from catalyst. The xylene solution was mixed with 180 mL of room temperature methanol to precipitate the extracted products which were separated from methanol via vacuum filtration. The products were analyzed by GPC. The results of the products formed in Example 15 are shown in Table 15 Table 3.

TABLE 14

| | | | Catalyst | Catalyst | | |
|---|---|---|---|---|---|---|
| | | Ethylene | Composition | Composition | Reaction | Reaction |
| | PE mass | mass | MTO/ | Cl—Al₂O₃ | temperature | time |
| Example | (mg) | (mg) | Cl—Al₂O₃ (mg) | (mg) | (° C.) | (hours) |
| 15-1 | 122 | 530 | 102 | 0 | 130 | 1 |
| 15-2 | 122 | 603 | 52 | 53 | 130 | 1 |
| 15-3 | 122 | 660 | 25 | 76 | 130 | 1 |
| 15-4 | 120 | 630 | 102 | 299 | 130 | 1 |

TABLE 15

| | Propylene | 2-butene |
|---|---|---|
| Example | produced (mg) | produced (mg) |
| 15-1 | 41 | 7.3 |
| 15-2 | 26 | 4.8 |
| 15-3 | 12 | 2.3 |
| 15-4 | 19 | 1.7 |

### Example 16. Catalytic Conversion of 1-Octadecene Over Catalyst Composition Example 1 in a Batch Reactor

In Example 16, 1-octadecene was reacted with ethylene over a mixture of MTO/Cl—Al₂O₃ and Cl—Al₂O₃, and in a batch reactor (Parr reactor, Series 2550) according to Table 6. 1-octadecene was degassed using three freeze-pump-thaw cycles and then transferred into a N₂-filled glovebox. The liquid was then dried with molecular sieves (3 Å). In an N₂-filled glovebox, the catalyst composition of Ex. 1 and 1-octadecene were loaded into a Parr reactor (10 mL, Series 2550) equipped with a pressure gauge and type K thermocouple. Ethylene (99.999%, Airgas) was passed through a moisture/oxygen trap (Supelco) before use. Gas lines were purged of residual air for three cycles before ethylene was introduced into the reactor. Heating to the designated temperature after pressurization with ethylene. After the designated reaction time, the reactor was cooled in flowing air. Aliquots of gas and liquids from the reactor headspace were taken for gas chromatography analysis with a flame ionization detector (GC-FID). The remaining solid and liquid were transferred onto a fine glass filter (4.0-5.5 μm) and filtered to remove insoluble material, such as coke, by washing with 5 mL CS₂ solution. After filtration, the filtrate solution was analyzed by GC-FID. The results of the products formed in Example 16 are shown in Table 17 Table 3.

TABLE 7

| | | | | MTO/ | | | |
|---|---|---|---|---|---|---|---|
| | 1- | | | Cl—Al₂O₃ | Cl—Al₂O₃ | | |
| | octadecene | Ethylene | Ethylene | Catalyst | Catalyst | Reaction | Reaction |
| | mass | mass | Pressure | mass | mass | temperature | time |
| Example | (mg) | (mg) | (psig) | (mg) | (mg) | (° C.) | (hours) |
| Ex. 16-1 | 200 | 511 | 657 | 101 | 103 | 100 | 1 |
| Ex. 16-2 | 200 | 507 | 652 | 104 | 202 | 100 | 1 |
| Ex. 16-3 | 200 | 505 | 649 | 107 | 105 | 100 | 1 |
| Ex. 16-4 | 200 | 505 | 648 | 100 | 100 | 100 | 5 |

TABLE 17

| | | | Mₙ of | M_{w} of |
|---|---|---|---|---|
| | Propylene | 2-butene | product | product |
| Tested | produced | produced | distribution | distribution |
| Property | (mg) | (mg) | (g/mol) | (g/mol) |
| Ex. 16-1 | 23 | 1.8 | 32 | 54 |
| Ex. 16-2 | 14 | 1.4 | 32 | 63 |
| Ex. 16-3 | 6.4 | 0.8 | 35 | 84 |
| Ex. 16-4 | 38 | 2.0 | 39 | 110 |

### Example 17. Catalytic Conversion of 1-Octadecene Using Alkene Reactants Other than Ethylene Over Catalyst Composition Example 1 in a Batch Reactor

In Example 17, 1-octadecene was reacted with propylene or 2-butene over the catalyst composition of Ex. 1 in a batch reactor (Parr reactor, Series 2550) according to Table 6. 1-octadecene was degassed using three freeze-pump-thaw cycles and then transferred into a N₂-filled glovebox. The liquid was then dried with molecular sieves (3 Å). In an N₂-filled glovebox, the catalyst composition of Ex. 1 and 1-octadecene were loaded into a Parr reactor (10 mL, Series 2550) equipped with a pressure gauge and type K thermocouple. Propylene (99.8%, Praxair) was passed through a moisture/oxygen trap (Supelco) before use. 2-butene (>99%, Sigma Aldrich) was used as received. Gas lines were evacuated to remove residual air before the alkene reactant was introduced into the reactor. Heating to the designated temperature occurred after pressurization with the alkene reactant. After the designated reaction time, the reactor was cooled in flowing air. Aliquots of gas and liquids from the reactor headspace were taken for gas chromatography analysis with a flame ionization detector (GC-FID). The remaining solid and liquid was transferred onto a fine glass filter (4.0-5.5 μm) and filtered to remove insoluble material, such as coke, by washing with 5 mL CS₂ solution. After filtration, the filtrate solution was analyzed by GC-FID. The results are shown in Table 19.

TABLE 18

| | 1- | | | | | | |
|---|---|---|---|---|---|---|---|
| | octadecene | Propylene | Butene | | Catalyst | Reaction | Reaction |
| | mass | Pressure | Pressure | Catalyst | mass | temperature | time |
| Example | (mg) | (psig) | (psig) | Composition | (mg) | (° C.) | (hours) |
| Ex. 17-1 | 200 | 179 | 0 | Ex. 1 | 101 | 100 | 1 |
| Ex. 17-2 | 100 | 16 | 0 | Ex. 1 | 63 | 100 | 1 |
| Ex. 17-3 | 100 | 0 | 4 | Ex. 1 | 60 | 100 | 1 |

TABLE 19

| | | | | | CS₂- | | |
|---|---|---|---|---|---|---|---|
| | | | | | soluble | Mₙ of | M_{w} of |
| | Ethylene | Propylene | 2-butene | Pentene | olefin | product | product |
| | produced | Produced | produced | produced | products | distribution | distribution |
| Example | (mg) | (mg) | (mg) | (mg) | (mg) | (g/mol) | (g/mol) |
| Ex. 17-1 | 88 | N/A | 18 | 0 | 143 | 53 | 118 |
| Ex. 17-2 | 1.2 | N/A | 7.3 | 0.8 | No data | No data | No data |
| Ex. 17-3 | 0.05 | 3.3 | N/A | 1.5 | No data | No data | No data |

### Example 18. Catalytic Conversion of 1-Pentene Over Catalyst Composition Example 1 in a Batch Reactor

In Example 18, 1-pentene was reacted with ethylene over a mixture of mixture of MTO/Cl—Al₂O₃ and Cl—Al₂O₃ in a batch reactor according to Table 6. 1-Pentene (Aldrich, 98.5%) was degassed using three freeze-pump-thaw cycles, vacuum distilled (ca. 10⁻⁴ torr), and transferred into a N₂-filled glovebox where the liquid was stored over molecular sieves (Aldrich, 3 Å, activated in vacuum overnight at 300° C.). Inside the glovebox, 1-pentene (1 mL) was sealed in a 1 mL Schlenk flask equipped with a Teflon stopper. A gas bulb partially-filled with molecular sieves (Aldrich, 3 Å, activated in vacuum overnight at 300° C.) was filled with 900 mbar ethylene containing 2 mol % propane as an internal standard (Praxair, UN1964, Lot No. 304325039305). The catalyst composition of Ex. 1 was loaded in a 120 mL Pyrex reactor in an Ar-filled glovebox. The reactor, ethylene gas bulb, and pentene flask were attached to a vacuum line. After evacuation of the connecting lines to remove air/water, the reactor was evacuated and heated to 100° C. The reactor was sealed, the connecting line was filled with ethylene. After the ethylene bulb was re-sealed (to prevent cross contamination), the 1-pentene storage flask was opened to produce a mixture of pentene and ethylene (after reaction, ethylene present in the 1-pentene flask was removed via freeze-pump-thaw cycles). The mixture of 1-pentene and ethylene (23 mol C₂/mol C₅) was admitted into the evacuated reactor creating a reactor pressure of 170 mbar. Aliquots of headspace gas were removed by gas-tight syringe at timed intervals by admitting a portion of the headspace gas into an evacuated section of the vacuum line, equipped with a rubber septum. Reaction progress was monitored by GC-FID (Shimadzu GC-2010, equipped with a Supelco Alumina Sulfate plot column, 30 m×0.32 mm), using the propane present in the ethylene as an internal standard. The column temperature was held constant at 90° C. After the reaction, nonvolatile products were recovered by extraction from the catalyst using room temperature CHCl₃. The extracted products were analyzed by GC-MS using a Shimadzu GC2010 gas chromatograph equipped with an Agilent DB-1 capillary column (dimethylpolysiloxane, 30 m×0.25 mm×0.25 μm) coupled to a QP2010 Mass Spectrometer. The injector and detector temperatures were 250° C. The temperature ramp program was: 40° C. (hold 3 min), ramp 25° C. per min to 250° C. (hold 10 min). The results are shown in Table 21.

TABLE 20

| | MTO/Cl—Al₂O₃ | Cl—Al₂O₃ |
|---|---|---|
| Example | mass (mg) | mass (mg) |
| 18-1 | 30 | 0 |
| 18-2 | 20 | 10 |
| 18-3 | 10 | 10 |
| 18-4 | 10 | 20 |
| 18-5 | 7.5 | 22.5 |
| 18-6 | 10 | 50 |

TABLE 21

| Tested | Initial Propylene Turnover Frequency |
|---|---|
| Property | (mol propylene h⁻¹ mol rhenium⁻¹) |
| 18-1 | 82 |
| 18-2 | 149 |
| 18-3 | 317 |
| 18-4 | 444 |
| 18-5 | 393 |
| 18-6 | 328 |

It is noted that one or more of the following claims utilize the term “where” or “in which” as a transitional phrase. For the purposes of defining the present technology, it is noted that this term is introduced in the claims as an open-ended transitional phrase that is used to introduce a recitation of a series of characteristics of the structure and should be interpreted in like manner as the more commonly used open-ended preamble term “comprising.” For the purposes of defining the present technology, the transitional phrase “consisting of” may be introduced in the claims as a closed preamble term limiting the scope of the claims to the recited components or steps and any naturally occurring impurities. For the purposes of defining the present technology, the transitional phrase “consisting essentially of” may be introduced in the claims to limit the scope of one or more claims to the recited elements, components, materials, or method steps as well as any non-recited elements, components, materials, or method steps that do not materially affect the novel characteristics of the claimed subject matter. The transitional phrases “consisting of” and “consisting essentially of” may be interpreted to be subsets of the open-ended transitional phrases, such as “comprising” and “including,” such that any use of an open ended phrase to introduce a recitation of a series of elements, components, materials, or steps should be interpreted to also disclose recitation of the series of elements, components, materials, or steps using the closed terms “consisting of” and “consisting essentially of.” For example, the recitation of a composition “comprising” components A, B, and C should be interpreted as also disclosing a composition “consisting of” components A, B, and C as well as a composition “consisting essentially of” components A, B, and C. Any quantitative value expressed in the present application may be considered to include open-ended embodiments consistent with the transitional phrases “comprising” or “including” as well as closed or partially closed embodiments consistent with the transitional phrases “consisting of” and “consisting essentially of.”

As used in the Specification and appended Claims, the singular forms “a”, “an”, and “the” include plural references unless the context clearly indicates otherwise. The verb “comprises” and its conjugated forms should be interpreted as referring to elements, components or steps in a non-exclusive manner. The referenced elements, components or steps may be present, utilized or combined with other elements, components or steps not expressly referenced.

It should be understood that any two quantitative values assigned to a property may constitute a range of that property, and all combinations of ranges formed from all stated quantitative values of a given property are contemplated in this disclosure. The subject matter of the present disclosure has been described in detail and by reference to specific embodiments. It should be understood that any detailed description of a component or feature of one or more embodiments does not necessarily imply that the component or feature is essential to the particular embodiment or to any other embodiment. Further, it should be apparent to those skilled in the art that various modifications and variations can be made to the described embodiments without departing from the spirit and scope of the claimed subject matter.

1. A process for converting unsaturated polyethylene to at least an alkene product of chemical formula CₘH₂ₘ, the process comprising contacting the unsaturated polyethylene with two or more catalyst components in a reactor, the reactor comprising an alkene reactant of chemical formula CₙH₂ₙ;
where:
m is an integer from 3 to 20
n is an integer from 2 to 20;
the two or more catalyst components comprise a metathesis catalyst component and an isomerization catalyst component; and
contacting causes at least a portion of the unsaturated polyethylene, or products derived therefrom, to undergo metathesis reactions and isomerization reactions to produce an effluent comprising at least the alkene product of chemical formula CₘH₂ₘ.

2. The process of any of claim 1, wherein a pressure of the alkene reactant in the reactor during the contacting is from 0 pounds per square inch gauge (psig) to 3000 psig.

3. The process of claim 1, wherein a temperature of the reactor during the contacting is less than or equal to 400° C.

4. The process of claim 1, wherein the alkene reactant comprises ethylene, propylene, butenes, pentenes, or combinations thereof.

5. The process of claim 1, wherein the alkene product comprises propylene, butenes, pentenes, or combinations thereof.

6. The process of claim 1, wherein the metathesis catalyst component comprises an element selected from International Union of Pure and Applied Chemistry (IUPAC) groups 5-10.

7. The process of claim 1, wherein the metathesis catalyst component comprises rhenium, ruthenium, tungsten, molybdenum, vanadium, or combinations thereof.

8. The process of claim 1, wherein the metathesis catalyst component comprises methyltrioxorhenium (MTO).

9. The process of claim 1, wherein the isomerization catalyst component comprises an element selected from IUPAC groups 5-10.

10. The process of claim 1, wherein the isomerization catalyst component comprises alumina, silica, iridium, palladium, ruthenium or combinations thereof.

11. The process of claim 1, wherein a first catalyst composition comprises the metathesis catalyst component and the isomerization catalyst component, and wherein the first catalyst composition comprises MTO on alumina.

12. The process of claim 1, wherein a first catalyst composition comprising MTO on alumina and a second catalyst composition comprising ruthenium, palladium, platinum, or combinations thereof contact the unsaturated polyethylene in the reactor.

13. The process of claim 1, wherein the process comprises contacting the unsaturated polyethylene with three or more catalyst components, and wherein the three or more catalyst components comprise a metathesis catalyst component, an isomerization catalyst component, and a dehydrogenation catalyst component.

14. The process of claim 13, wherein the dehydrogenation catalyst component comprises an element selected from IUPAC groups 5-10.

15. The process of claim 13, wherein the dehydrogenation catalyst component comprises platinum, iridium, ruthenium, rhenium, or combinations thereof.
